(19) United States
(12) Plant Patent Application Publication
(10) Pub. No.: US 2002/0104133 P1
(43) Pub. Date: Aug. 1, 2002

(54) Shrub rose plant named 'CHEwily'

(76) Inventor: Chris Warner, Greenfields Brockton (GB)

Correspondence Address: CHRISTIE, PARKER & HALE, LLP, 350 WEST COLORADO BOULEVARD, SUITE 500, PASADENA, CA, 91105, US

(21) Appl. No.: 09/771,903

(22) Filed: Jan. 29, 2001

(51) Int. Cl. A01H 5/00

(52) U.S. Cl. PLT/107

(57) ABSTRACT

A new variety of Shrub rose suitable for garden decoration, having flowers of pastel pink coloration.

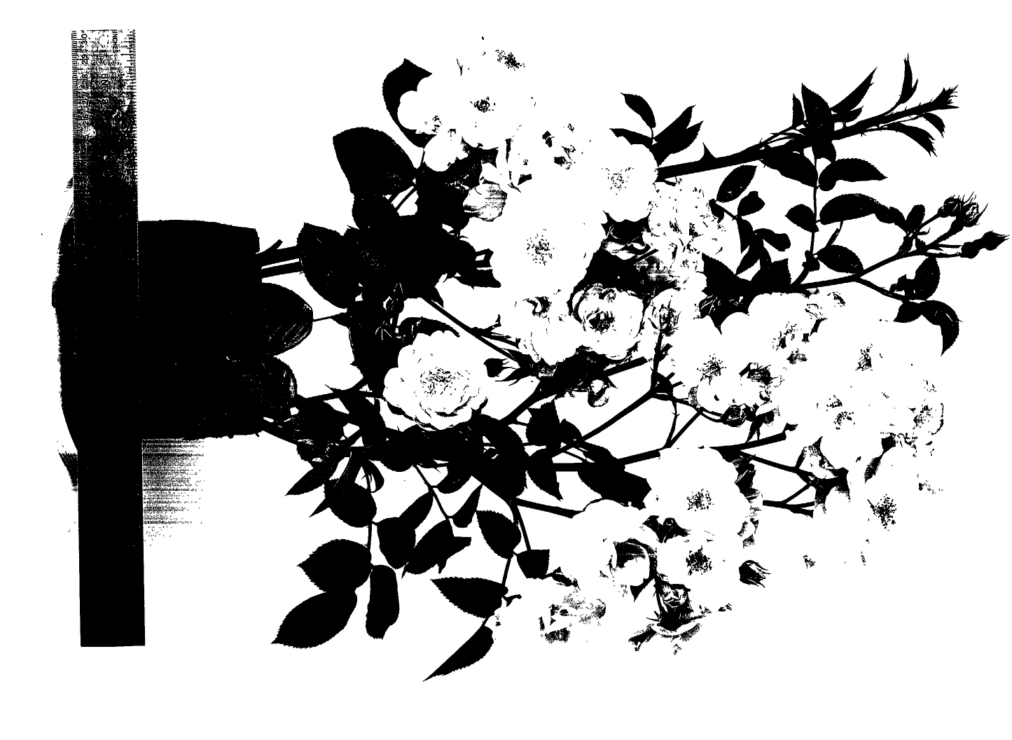

### BACKGROUND OF THE INVENTION

This invention relates to a new and distinct variety of Shrub Rose. The varietal denomination of the new variety is ‘CHEwily’. It has as its seed parent the variety known as Seaspray and as its pollen parent an undistributed seedling.

### SUMMARY OF THE INVENTION

Among the features which distinguish the new variety from other presently available and commercial rose cultivars known to the inventor are the following combination of characteristics: its small somewhat globular bud with a conspicuous hypanthium, its hairy peduncule, its continuity of color from the fresh to the old flower, its glossy foliage often suffused with dark red pigmentation and its dark red rachis. The plant has a rounded to slightly spreading growth habit, suitable for garden decoration.

Asexual reproduction of the new variety by budding as performed in Kern County and Upland, Calif., shows that the foregoing and other distinguishing characteristics come true to form and are established and transmitted through succeeding propagations. ‘CHEwily’ may be asexually propagated by cuttings, budding and grafting.

### Comparison with Parents

The new rose may be distinguished from its seed parent, ‘Seaspray’ by the following combination of characteristics: whereas ‘CHEwily’ bears flowers of pastel pink coloration, ‘Seaspray’ bears flowers of pastel pink coloration often blushed with red and with significantly more yellow tones at the petal base. The new variety bears glossy foliage often suffused with dark pigments whereas the seed parent bears foliage with a matte finish and significantly less suffusion of darker pigments.

The new variety may be distinguished from its pollen parent, an undistributed seedling, by the following combination of characteristics: whereas ‘CHEwily’ produces flowers of semi-double petalage (about 14 to about 16 petals), the pollen parent produces flowers with significantly less petalage (about 5). The pollen parent has a very large somewhat climbing mature growth habit with large foliage, whereas the new variety has a significantly smaller more compact mature growth habit with smaller foliage.

### BRIEF DESCRIPTION OF ILLUSTRATION

The accompanying photograph illustrates specimens of the new variety and shows the flowering thereof from bud to full bloom depicted in color as nearly correct as it is possible to make in a color illustration of the character. Throughout this specification, color values are based upon the Colour Chart of The Royal Horticultural Society of London, England, except where common terms of color definition are employed.

### DESCRIPTION OF THE NEW VARIETY

The following description is of rose plants of the new cultivar grown outdoors in Upland, Calif. in the month of October. Phenotypic expression may vary with environmental, cultural and climatic conditions, as well as differences in conditions of light and soil.

### FLOWER

The new variety usually bears large clusters with several flowers per stem. Flowers are borne in irregular rounded to moderately pyramidal clusters on strong long stems (about 26 to about 68 cms.) that are sometimes pendulous with the weight of the cluster. Outdoors, the plant blooms abundantly during the growing season. The flowers have a very slight tea fragrance.

### BUD

The peduncle is about 1.2 to about 3.2 cms. in length, of slender caliper and usually erect. It is moderately smooth, with some stipitate glands and many hairs. Peduncle color is between 139A and 144A often heavily suffused especially on the side exposed to the sun with between 183A and 187B.

Before the calyx breaks, the bud is about 0.7 to about 1.2 cms. in diameter at the widest point, about 0.8 to about 1.1 cms. in length and pointed to somewhat globular in shape with a large conspicuous hypanthium. The surface of the bud bears few foliaceous appendages and some stipitate glands, usually with slender entire foliaceous parts extending beyond the tip of the bud about ¼ of its length. Bud color is between 139A and 144A often heavily suffused with between 187A and 183A.

The inner surface of the sepals is covered with fine wooly tomentum; sepal margins are lined with many stipitate glands and hairs.

As the petals open (after the calyx breaks), the bud is about 1.0 to about 1.5 cms. in diameter at the widest point, about 1.1 to about 1.4 cms. in length and moderately ovoid to somewhat globular to slightly pointed in form. The color of the under surfaces of the newly opened petals is between 52B and 55B. At the point where the petal attaches, there is a moderately small zone of between 10B and 12C. The color of the upper surfaces of the newly opened petals is between 38B and 55C. At the point where the petal attaches, there is a moderately small zone of between 13C and 12B.

### BLOOM

When fully open, the bloom ranges from about 4.0 to about 5.6 cms. in diameter. Petalage is semi-double with about 14 to 16 petals and about 1 to 2 petaloids arranged irregularly. When partially open, the bloom form is very cupped and the petals are loosely cupped to slightly undulated with petal edges slightly reflexed outward. When fully open, the bloom form and the petals are less cupped to moderately flat with petal edges slightly reflexed outward.

### PETALS

The substance of the petals is moderate and somewhat thin, with upper surfaces moderately satiny and under surfaces somewhat satiny to shiny. The outer petals are somewhat round to very broadly obovate in shape with rounded apices. The inner petals are more obovate in shape with rounded apices.

### NEWLY OPENED FLOWER

The under surface of the outer and intermediate petals is between 62C and 62A. The under surface of the inner petals is between 62D and 62C. At the point where the petal attaches, there is a small zone of between 8D and 10D.

The upper surface of the outer, intermediate and inner petals is between 62C and 62D. At the point where the petal attaches, there is a small zone of between 8C and 10C.

The general tonality of the newly opened flower is between 62C and 62D.

### THREE-DAY-OLD FLOWER

The under surface of the outer and inner petals is between 62C and 62B. At the point where the petal attaches, there is a small zone of near 11D.

The upper surface of the outer and inner petals is between 62C and 62D. The upper surface of the outermost petals is sometimes blushed with between 59C and 60C. At the point where the petal attaches, there is a small zone of between 11D.

The general tonality of the three-day-old flower is between 62C and 62D.

On the spent bloom, the petals usually drop off cleanly.

In October in Upland, Calif., blooms on the bush growing outdoors generally last about three to five days. Cut roses from plants grown outdoors and kept at normal indoor living temperatures generally last about three to five days.

### MALE REPRODUCTIVE ORGANS

Stamens are average in number (average about 90) and are arranged regularly about the pistils; a few are mixed with petaloids. The filaments are moderately long to somewhat irregular in length, most with anthers. Filaments are between 9C and 13B in color. The anthers are moderately small for the class and they all open approximately at the same time. Anther color is between 22A and 15B when immature. Anther color at maturity is as dark as 165A sometimes marked irregularly with near 22A. Pollen is somewhat sparse and near 11D in color.

### FEMALE REPRODUCTIVE ORGANS

Pistils vary in number (average about 45). The styles are somewhat even and loosely bunched. Stigma color is between 24B and 22A. Style color is between 2D and 4D sometimes lightly blushed with near 52B. Ovaries are usually all enclosed in the calyx.

Hips are of short to average length, very globular in form and near 28A in color when ripe. The hip surface is very smooth with moderately thick fleshy walls. The sepals are moderately fugacious. There are approximately 2 to 8 seeds, moderately small in size.

### FOLIAGE

The compound leaves are usually comprised of three to seven or more leaflets and are borne abundantly. The seven-leaflet leaves are about 8.8 to about 12.7 cms. in length and about 7.6 to about 9.4 cms. in width at the widest point, moderately thick, somewhat crisp in texture and very glossy in finish. The terminal leaflets are about 4.0 to about 6.1 cms. in length and about 2.2 to about 3.8 cms. in width at the widest point, shaped oval to somewhat ovate with moderately acute to somewhat acuminate apices and round bases. Their margins are usually simply serrate.

The upper surface of the mature leaf is between 133A and 137A. The under surface of the mature leaf is between 147B and 138B. The upper and under surface of the young leaf is between 144A and 137B usually heavily suffused with between 187A and 183A.

The rachis is light in caliper and somewhat rough. The upper side is shallowly grooved with some hairs on the edges of the grooves. The under side of the rachis is somewhat rough with few small prickles and stipitate glands. On the younger growth, the rachis is often heavily suffused with between 187A and 183A.

The stipules are about 1.0 to about 1.5 cms. in length and of medium width with straight points that usually turn out at an angle of more than 90 degrees.

The plant displays an average degree of resistance to powdery mildew and rust as compared to other commercial varieties grown under comparable conditions in Upland, Calif.

### GROWTH

The plant has a rounded to slightly spreading medium height growth habit (about 110 to about 125 cms. in height and about 160 to about 190 cms. spread at the widest point), with full branching. It displays very vigorous growth and the canes are of medium to heavy caliper for the class.

The color of the major stems is between 146B and 144A. They bear many large prickles that are about 0.7 to about 1.1 cms. in length. The large prickles are hooked moderately downward with a long oval base; prickle color is between 165B and 166B. The major stem bears few small prickles of similar shape and coloration.

The color of the branches is between 136B and 144A sometimes heavily suffused on the side exposed to the sun with between 187B and 183B. They bear few large prickles that are of similar size and shape to the large prickles on the major stems; prickle color is between 166C and 166B. The branches bear few small prickles of similar shape and coloration.

The color of the new shoots is between 144A and 137B often heavily suffused with between 187A and 183A. They bear many large prickles which are of similar size and shape to the large prickles on the major stems; prickle color is between 187B and 183B. The shoots bear few small prickles of similar shape and coloration to the large prickles of the new shoots.

I claim:

1. A new and distinct Shrub rose plant of the variety substantially as described and illustrated herein.
